(19) United States
(12) Patent Application Publication
(10) Pub. No.: US 2007/0066934 A1
(43) Pub. Date: Mar. 22, 2007

(54) Electrokinetic delivery system and methods therefor

(75) Inventors: Robert W. Etheredge III, Natick, MA (US); Dennis I. Goldberg, Waltham, MA (US); Phillip M. Friden, Bedford, MA (US); John S. Peterson, Acton, MA (US)

Correspondence Address: NIXON & VANDERHYE, PC, 901 NORTH GLEBE ROAD, 11TH FLOOR, ARLINGTON, VA, 22203, US

(73) Assignee: Transport Pharmaceuticals, Inc., Framingham, MA (US)

(21) Appl. No.: 11/228,461

(22) Filed: Sep. 19, 2005

(51) Int. Cl. A61B 17/20

(52) U.S. Cl. 604/46; 604/173

(57) ABSTRACT

The electrokinetic medicament delivery system includes at least one applicator having a multiplicity of non-conductive micro-needles carried on a non-conductive surface of the applicator. The opposite surface is formed of electrically conductive material for contact with an active electrode. The applicator includes a matrix containing a medicament or a carrier therefor between the opposite surfaces. When the applicator is applied to the individual's skin with the micro-needles penetrating the skin, an electrical current is completed through the power source, the active electrode, medicament, or electrically conductive carrier therefor, the targeted treatment site, the individual's body, a ground electrode and the power supply, thereby electokinetically driving the medicament through the non-conductive micro-needles into the targeted treatment site.

### BACKGROUND OF THE INVENTION

The present invention relates generally to the electrokinetic mass transfer of substances into and/or extracting substances from tissue and particularly to apparatus and methods for delivering substances, e.g., a medicament to a treatment site.

Electrokinetic delivery of medicaments for applying medication locally through an individual's skin is known. One type of an electrokinetic delivery mechanism is iontophoresis, i.e. the application of an electric field to the skin to enhance the skin's permeability and to deliver various ionic agents, e.g., ions of soluble salts or other drugs into the skin. In certain situations, iontophoretic transdermal or transmucosal delivery techniques have obviated the need for hypodermic injection for many medicaments, thereby eliminating the concomitant problem of trauma, pain and risk of infection to the individual. Other types of electrokinetic delivery mechanisms include electroosmosis, electroporation, electromigration, electrophoresis, and endosmosis, any or all of which are generally known as electrotransport, electromolecular transport or iontophoretic methods.

In recent years, various mechanisms for electrokinetically delivering a substance, e.g., a medicament to a treatment site include, for example, a finger mounted electrokinetic delivery system for self-administration of medicaments as disclosed in U.S. Pat. No. 6,792,306, of common assignee herewith, the disclosure of which is incorporated herein by reference. That system includes a power source, active and ground electrodes and a medicament containing matrix whereby, upon application of the active electrode to the treatment site, an electrical circuit is established from the power source, through the medicament or a conductive carrier therefor, the treatment site, the individual's body and the ground electrode to drive the medicament into the treatment site. Other electrokinetic delivery mechanisms are set forth in U.S. Pat. No. 6,895,271, issued May 17, 2005; U.S. Pat. No. 6,735,470, issued May 11, 2004; U.S. Pat. No. 6,477,410, issued Nov. 5, 2002 and U.S. Reissue Pat. No. RE 37796, re-issued Jul. 23, 2002, the disclosures of which are also incorporated herein by reference.

While those systems have been found to be efficacious, it will be appreciated that an individual's skin is formed of many different layers e.g. the Epidermis and the Dermis, both of which overlie the subcutaneous cellular tissue and each of which are, in turn, formed of various sub-layers. Of particular significance is the epidermis which is non-vascular and consists of stratified epithelium including the stratum corneum with various underlying sub-layers. These layers offer various electrical resistances to penetration of electrokinetically driven substances through the skin to a targeted layer. For example, the outer stratum corneum layer, offers very high electrical resistance to electrokinetic delivery of a substance through that layer into the underlying sub-layers. High electrical resistance impedes the electrokinetic delivery of the substance to the targeted site. The amount of medicament delivered across an individual's skin is dependent, in part, upon current density. As the area of iontophoretic treatment expands, total current increases to maintain the prescribed current density. For example, if a current density of 250 μA/cm² is prescribed for delivery of a specific medicament and the area of the iontophoretic delivery system is 4 cm², total current will be 4×250 μA or 1 mA. If the area of the iontophoretic delivery system is increased to 100 cm², total current would have to be 25 mA to maintain current density. Administration of this level of current presents a potential risk of damaging the patient's skin.

A further significant problem for electrokinetically driving substances through the skin includes the use of multi-channel electrodes, i.e., an array of individualized electrodes, each connected to a discrete donor site of medicament thereby creating individually controlled electric fields for larger area electrokinetic application of the medicament to the skin. For example, when a multi-channel electrode device is placed in contact with the skin in the presence of a conductive liquid, e.g., the medicament or a conductive gel and the liquid crosses over between electrodes, a short circuit may occur that compromises the multi-channel device. If a unified field is created and if there is an area of low resistance, there is the likelihood that the current will be channeled into that low resistance area, possibly burning the individual's skin. This has been a limiting factor in large area electrokinetic application of substances through an individual's skin. Consequently, there is a need to provide systems and methods for facilitating electrokinetic penetration of larger areas of an individual's skin in a manner which is not adversely affected by high electrically resistant layers of the skin while minimizing or eliminating short circuiting of the current as the substance is transported electrokinetically through the skin to the targeted site.

### DESCRIPTION OF EXAMPLE EMBODIMENTS

In accordance with example embodiments of the present invention, there are provided systems and methods for penetrating a high electrically resistant layer(s) of the skin, e.g., the stratum corneum to create an electrical connection directly between the active electrode through the drug-filled matrix into the targeted site, e.g., the epidermal layer, bypassing the high resistant skin layer. It will be appreciated that the epidermal layer of the skin below the stratum corneum has a high fluid content that is also conductive which provides a much larger receptor area for the supplied substance as compared with higher electrically resistant layers, such as the stratum corneum. To penetrate one or more high electrically resistant layers to supply medicament to a targeted underlying layer or layers, a pad or applicator is provided having a surface array of needles, preferably micro-needles along one side or face of the applicator. The needles are carried by a non-conductive membrane of the applicator and project from the membrane a distance sufficient to penetrate the high electrically resistant layer(s), upon application of the applicator to the individual's skin. Because of the very high density of the needles, preferably micro-needles, numerous low electrically resistant areas are created by perforating the high electrically resistant layer(s). That is, the needles form a multiplicity of channels i.e., micro-channels through the more highly electrically resistant layer(s). The needles in effect create channels in the skin. The length and density of the needles as well as the thickness or diameter of the needles including the diameter of the orifices through the needles can be varied depending upon the location of the targeted treatment site underlying the skin surface. The needles may be formed of a non-conductive material, e.g., a plastic material or may be formed of metal material coated with a non-conductive material. The needles can be monolithic with well-defined orifices for delivery of actives or fused particulates (sintered) that provide a porous needle with a tortuous network of many liquid transport paths in a more tortuous design. Such sintered material avoids the problem of needle coring of stratum-corneum tissue that occludes the fluid passages. It is understood that such material would include filaments, particles, staple fibers, wires or other forms of needle material that is joined under pressure to create a porous needle structure. Needles may also be made of conductive materials and coated with nonconductive layers. The needles may also be made of non-conductive intermetallic glasses. The needles may also be formed of bioresorbable polymers containing drugs or other active ingredients molecularly dissolved or dispersed as a separate phase. The active ingredient is delivered to the skin electrokinetically as the needle polymer is eroded and/or solubilized by interstitial fluid within the skin. Polymers such as polylactic acid, polyglycolic acid, copolymers of poly(lactide-glycolide), polyorthoesters, polyvinylalcohol and others, as well as natural products such as sugars, starches and graft copolymers of these. The opposite side of the pad from the needles may comprise a conductive membrane in contact with an active electrode and a power supply.

The micro-needles may be attached to a flexible substrate to provide a compliant system for skin interface. Micro-needles may not penetrate the epidermis to the full extent of needle height due to the compliant nature of the stratum-corneum and dermal underlayers. Additionally, skin is a viscoelastomer that relaxes mechanically under load. This causes the substrate to move away from the needle during puncture. One means for improving the consistency of puncture by needle arrays is to impose an upward movement of the skin using an iontophoretic patch. The patch may include a rigid boundary surrounding an array of micro-needles enabling, upon application, the skin surrounded by the boundary to present itself, i.e., become proud of skin adjacent the patch, to the micro-needle array. In another embodiment, to provide skin penetration, the arrays of micro-needles are attached to a slightly concave-shaped elastomeric backing attached to the iontophoretic patch and acts as a suction cup. Upon actuation by the user, the target skin area is pilled into the concavity and against the micro-needles attached to the more rigid backing material. Micro-needles are thus allowed to penetrate the skin without interference from the more compliant dermal layers below.

The system also includes a device containing the active and ground electrodes and a power supply. Preferably, the applicator and the device are separable from one another whereby the applicator is disposable and the device may be reused with a fresh applicator. Alternatively, the device and applicator may constitute an integrated disposable or reusable unit.

In another embodiment hereof, groups of the applicators may be provided, for example, on sheet material whereby the applicators are separable, e.g., by perforation lines through the sheet. Thus, the involved area of the applicator overlying the treatment site can be varied in size. A multi-channel electrode array is therefor coupled to the applicators whereby the area coverage of the applicators can be personalized to the size of the targeted treatment site. It will be appreciated that the shape of the applicators can vary, e.g., circular, rectilinear, hexagonal or any other shape. In this manner, the needles provide multiple very low electrically resistant pathways through the high electrically resistant layer(s) enabling, for example a micro-processor to drive via the multi-channel electrode array the medicament or a carrier therefor disposed in a matrix within the applicator through the skin to apply the medicament directly to the targeted treatment site.

As noted previously, the applicator containing the needles may be combined with a delivery device. For example, the finger mounted devices disclosed in U.S. Pat. Nos. 6,792,306 and 6,735,470, may be provided with applicators containing needles of selected sizes and configurations to penetrate through the high electrically resistant layers of the skin to supply medicament to the targeted treatment site. Alternatively, the device disclosed in U.S. Pat. No. RE37796, may likewise use applicators of the type described herein. In all instances, by forming a multiplicity of low electrically resistant perforations or pathways through the higher electrically resistant layer or layers of the skin, the substance can be driven from the supply matrix through the needles directly to the targeted treatment site bypassing the high electrically resistant skin layer(s).

Advantages of using the present delivery system include the capacity to increase the quantity of the substance delivered by reducing the resistance to penetration of the substance through the skin. The provision of multiple pathways, e.g., micropores enables delivery of an array of drugs, e.g., large molecules such as peptides, liposomes encapsulating hydrophobic drugs, or other encapsulated drug formulations not currently deliverable by electokinetic processes, particularly iontophoresis. Further, by controlling the length of the needles, the substance may be delivered to selective targeted sites at different skin depths. For example, if just the stratum corneum is penetrated, the underlying layers of the epidermis are used as a substance reservoir with that area being loaded with the substance bypassing the stratum corneum and enabling administration of the substance. Further penetration by the needles enables proximity to the blood supply enabling systemic administration of substances making the electrokinetic process appropriate for delivery of systemic drugs. Also, by locating the substance supply close to the blood supply, the substance can clear its entry points quickly enabling substance delivery on a more continuous basis.

In a preferred embodiment of the present invention, there is provided a device for delivering a medicament to a treatment site underlying an electrically resistant layer of an individual's skin, comprising an applicator for overlying the treatment site and the electrically resistant skin layer, the applicator having a plurality of needles projecting from a first surface thereof for penetrating the electrically resistant layer of the individual's skin, the needles and the surface being formed of a non-electrically conductive material; a matrix carried by the applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; the applicator having a second surface formed of electrically conductive material.

In a further preferred embodiment, there is provided a system for delivering a medicament to a treatment site underlying an electrically resistant layer of an individual's skin, comprising an applicator for overlying the treatment site and the electrically resistant skin layer, the applicator having a plurality of needles projecting from one side thereof for penetrating the electrically resistant layer of the individual's skin; a matrix carried by the applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; a first electrode for electrical connection with a power source; whereby, upon application of the applicator to the individual's skin overlying the treatment site and connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow for electrokinetically driving the medicament or the medicament and the electrical carrier therefor through the needle orifices into the treatment site bypassing the electrically resistant layer of the individual's skin.

In a still further preferred embodiment, there is provided a system for delivering a medicament to a treatment site underlying an electrically resistant layer of an individual's skin, comprising a power source; an applicator for overlying the treatment site and the electrically resistant skin layer, the applicator having a plurality of needles projecting from one side thereof for penetrating the electrically resistant layer of the individual's skin; a matrix carried by said applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; a first electrode carried by the applicator in electrical connection with the power source; a second electrode in electrical connection with the power source; whereby, upon application of the applicator to the individual's skin overlying the treatment site and electrical connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow to electrokinetically drive the medicament or the medicament and the electrical carrier therefor through the needle orifices into the treatment site bypassing the electrically resistant layer of the individual's skin.

Another preferred embodiment of the present invention includes a system for delivering a medicament to a treatment site underlying an electrically resistant layer of an individual's skin, comprising a sheet of discrete applicators selectively separable from one another enabling one or more of the applicators to overlie the treatment site and the electrically resistant skin layer, each applicator having a plurality of needles projecting from one side thereof for penetrating the electrically resistant layer of the individual's skin; a matrix carried by each applicator for containing the medicament or the medicament and an electrical carrier therefor, the needles of each applicator having one or more orifices in communication with the medicament or the medicament and the electrical carrier therefor contained in the matrix and opening at locations spaced from the matrix for delivering the medicament to the treatment site; a first electrode carried by each applicator for electrical connection with a power source; whereby, upon application of one or more of the applicators to the individual's skin overlying the treatment site and connection to the power source and a second electrode in electrical connection with the power source enabling completion of an electrical circuit through the first one or more electrodes, the medicament or the electrical carrier therefor of the one or more applicators, a portion of the individual's body, the second electrode and the power source, the system enables an electrical current to flow for electrokinetically driving the medicament or the medicament and the electrical carrier therefor through the needle orifices of the one or more applicators into the treatment site bypassing the electrically resistant layer of the individual's skin.

In a still further embodiment hereof, there is provided a method for delivering medicament to a treatment site underlying an electrically resistant layer of an individual's skin, comprising the steps of applying a plurality of micro-needles to the individual's skin to penetrate the electrically resistant layer of the individual's skin; and electrokinetically driving the medicament or the medicament and an electrical carrier therefor through the micro-needles into the treatment site bypassing the electrically resistant layer of the individual's skin.

### BRIEF DESCRIPTION OF THE DRAWINGS

FIG. 1 is a schematic illustration of an electrokinetic substance delivery applicator in accordance with a preferred embodiment of the present invention;

FIG. 2 is a schematic illustration of a multi-channel electrode array under microprocessor control and illustrating a plurality of applicators each containing a multiplicity of needles;

FIG. 3 is a view similar to FIG. 2 illustrating a further embodiment of the present invention; and

FIG. 4 is a schematic view of a pair of applicators arranged side by side for larger area coverage;

FIG. 5 is a schematic representation of various micro-needle structures with one or more orifices, sizes and locations;

FIG. 6 is a fragmentary enlarged view illustrating an applicator with micro-needles penetrating different portions of an individual's skin;

FIG. 7 is a fragmentary perspective view illustrating the underside of an applicator using clusters of micro-needles and discrete electrode channels; and

FIG. 8 is a schematic illustration of a specific application in accordance with an embodiment of the present invention.

### DETAILED DESCRIPTION OF EXAMPLE EMBODIMENTS

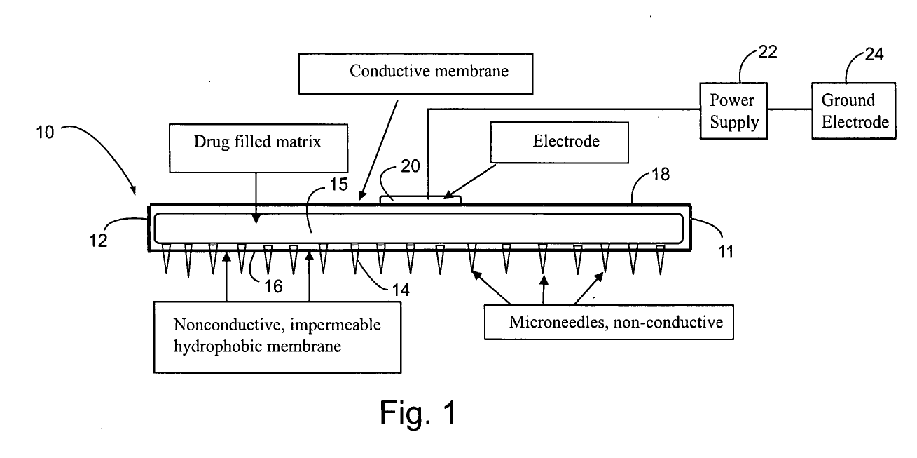

Referring to the drawings, particularly to FIG. 1, there is illustrated a system for delivering a medicament to a treatment site underlying one or more high electrically resistant layers of an individual's skin. The system, generally designated 10, includes an applicator 11 comprising an enclosure 12 housing a matrix 14 containing a medicament, such as acyclovir or a carrier therefor. The term medicament is used in a broader sense synonymous with the term substance and therefore embraces natural or homeopathic products that may be outside the standard definition of a medicament, e.g., inks and pigments for tattoos and more generally includes any substance capable of electrokinetic transport through skin or mucocutaneuous membrane into or from a treatment site for multiple purposes, e.g., diagnostic or treatment purposes. Thus, by medicament is meant any chemical or biologic substance that may be used on or administered to humans or animals as an aid in the diagnostic treatment or prevention of disease or other abnormal or cosmetic condition or for the relief of pain or to control, diagnose, measure detoxify or improve any psychological or pathologic condition. Since the majority of applications using the present invention are for applying medicaments to treatment sites, the term “medicament” is used throughout and includes the more general term “substance”. By a treatment site is meant any target tissue, e.g., a diseased tissue or diagnostic/detoxification site for extraction or application of a substance, underlying or exposed through or on an individual's skin, cutaneous or mucocutaneous membrane. Also, certain medicaments are not electrically conductive. To electrokinetically drive such medicaments, an electrically conductive carrier is provided the medicament to carry the medicament into the treatment site.

The applicator 11 includes a multiplicity of needles 14, preferably micro-needles projecting from one side of the housing 12. The needles 14 are carried by, and penetrate through, a non-conductive impermeable, preferably hydrophobic membrane 16 along the face of the applicator which is to be applied in overlying relation to the skin and hence the treatment site. By preferably using a hydrophobic membrane, movement of liquid at the interface is resisted and which otherwise might act to bridge individual channels. The non-conductive impermeable membrane 16 has edges along the margins of the applicator which are likewise non-conductive and impermeable. The opposite face of the applicator 11 is formed of a conductive membrane 18. A drug-filled matrix 15 is sandwiched between the impermeable membrane 16 and the conductive membrane 18, so that the matrix and drug contained within are contiguous with the bases of the needles 14 and particularly the orifices through the needles are described below. A first or active electrode 20 is illustrated in electrical contact with the conductive membrane 18 and with a power supply 22. Also connected to the power supply is a second or ground electrode 24 for application to another part of the individual's body spaced from the targeted treatment site. The ground electrode 24 completes the electrical circuit for the electrokinetic delivery of the medicament to the targeted treatment site as described below.

The needles 14 are preferably micro-needles formed of a non-conductive material, such as a thermoplastic material, e.g., a polycarbonate, polyester, polymethylacrylate or other materials sufficiently rigid to penetrate the skin when applied to the skin. The micro-needles may also be formed of thermoset materials, such as epoxy, polyurethane and silicones. The micro-needles may also be formed of metal materials coated both externally and internally with a non-conductive material, such as a thermoplastic and which may be polymeric in nature or inorganic, such as oxide layers. The micro-needles 14 have a density in the range of about 1-1000 needles per cm², and preferably in a range of about 150-250 needles per cm². The height of the needles 14 projecting from the non-conductive membrane 16 may lie within a range of 100 to 800 microns. The micro-needles are preferably conically or pyramidally shaped and have a height equal to about twice the diameter of the base. The base can be nominally one-half the height to about twice the height Thus, for example, a needle 400 microns in height may have a base of about 200 microns. For the same needle, the orifice through the needle may have a diameter in a range of 25-200 microns. The micro-needles may also have a constant width throughout their length in contrast to the preferred conical or pyramidal shape. Thus, each micro-needle may have less than one millimeter in length, be useful to penetrate the uppermost layers of tissue such as the stratum corneum of human skin, may contain one or more conduits for passage of liquids between interstitial regions of the tissue and a medical or drug-delivery device may be comprised of or coated with nonconductive materials to allow for electrokinetic transport of ions through the micro-needle.

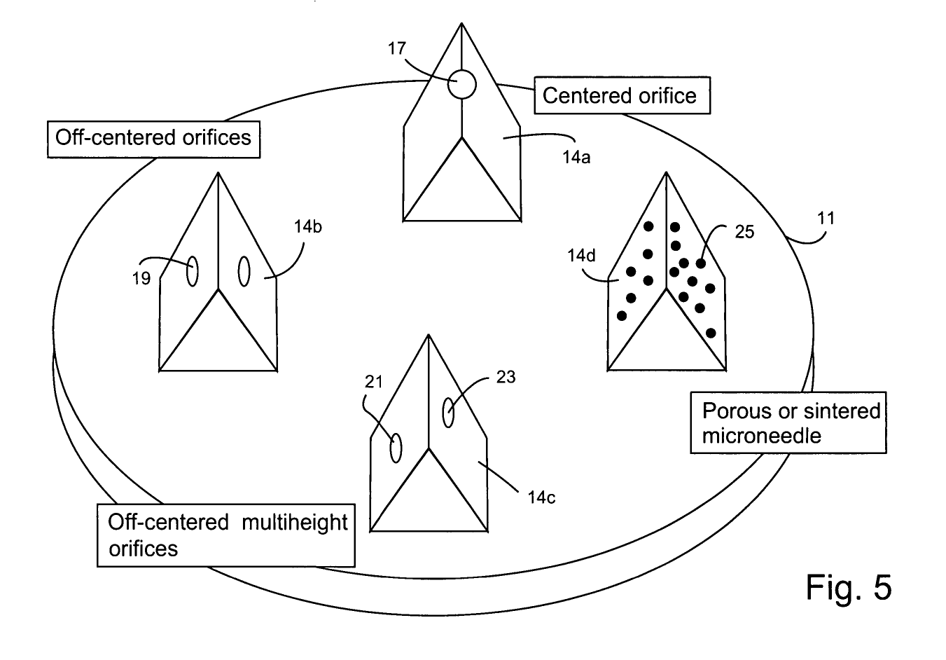

Referring to FIG. 5, there is schematically illustrated various micro-needle structures forming part of an applicator. For example, the micro-needle 14a may have an orifice 17 centered along the height of the micro-needle. Micro-needle 14b includes a plurality of orifices 19 located off the axial center of the micro-needle. The orifices 19 may individually lie in communication with the drug-filled matrix 15 or lie in communication with a single passage in communication with matrix 15. Micro-needle 14c may include off-centered multiple height orifices 21 and 23 and consequently, delivery of a medicament may occur at different depths within the individual's skin by way of a single micro-needle. Combinations of centered, off-centered and multiple height or depth orifices may also be provided in a single micro-needle. Micro-needle 14d may comprise a micro-porous structure having a multiplicity of micro-pores 25. The micro-needle 14d may be comprised of a sintered material to create a network of tortuous channels in communication with the drug-filled matrix 15. Combinations of the various types of micro-needles disclosed in FIG. 5 may also be utilized in a single applicator.

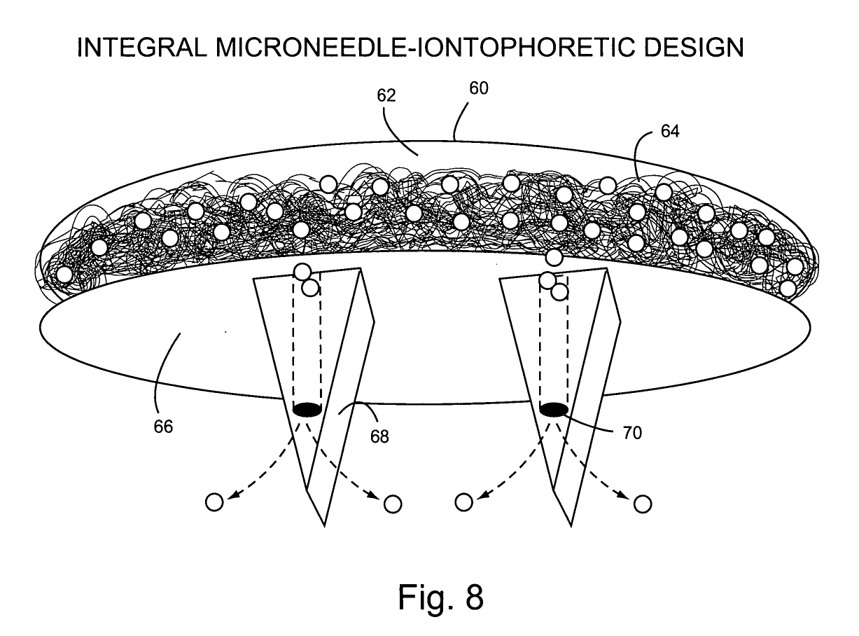

In FIG. 1, the applicator 11 may be separable from or an integral part of an applicator device such as disclosed in the aforementioned patents. Thus, in one embodiment, the applicator 11 may form a disposable part of the device while the electrode, power supply, ground electrode and other electronics may form part of a reusable device. For example, the applicator 11 may comprise the substrate containing the medicament in the finger mounted device of FIGS. 8 and 9 of U.S. Pat. No. 6,792,306, or the hand-held pen-like and other devices of U.S. Pat. Nos. 6,477,410 and RE37796.

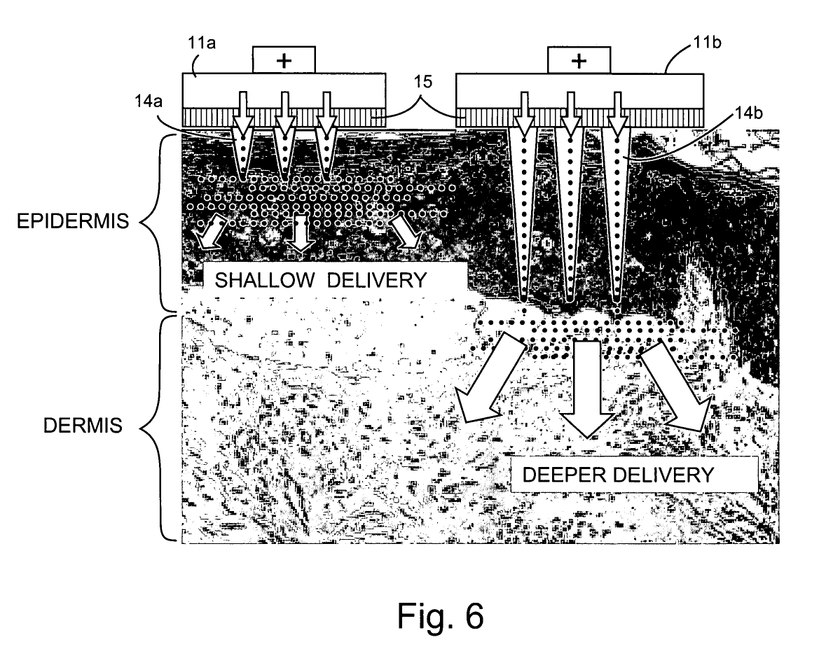

In an illustrative embodiment of the invention, for example, for supplying medicament to a targeted treatment site underlying one or more layers, e.g., the stratum corneum of the skin, an applicator is selected having needles 14 of appropriate size and configuration, e.g., length, width, orifice depth and orifice size, to penetrate the stratum corneum with the tip of each needle being exposed in the targeted layer. Thus, the targeted layer could be any sub-layer under the stratum corneum, i.e., any layer of the epidermis or layers of the dermis or below. For example and referring to FIG. 6, the applicator 11a may have relatively short micro-needles 14a for penetration of the epidermis and consequently a shallow delivery of the medicament into the epidermis. The other applicator 11b, illustrated in FIG. 6, may have longer micro-needles 14b for a deeper delivery of the medicament, e.g., at the beginning of the dermis. In both applicators of FIG. 6, the medicament is referenced by the arrows showing the direction of the delivery and the small black dots illustrate the respective areas of the epidermis and dermis into which the medicament is electrokinetically driven by applicators 11a and 11b. Consequently, an applicator containing the appropriate needle size and configuration to supply medicament directly to the intended treatment site at a predetermined depth below the exposed surface of the skin would be selected. It will be appreciated that, with the needles forming a multiplicity of non-conductive pathways through the selected layer or layers of the skin and affording direct communication of the medicament or carrier therefor from the medicament-filled matrix 15 through the needle orifice to the treatment site, i.e., the target layer, activation of the electrokinetic device drives the medicament from the matrix through the needles into the targeted layer. That is, with the ground electrode in electrical contact with the individual's body at a location spaced from the treatment site and the power supply in an “on” condition, an electrical circuit is completed from the power supply 22, through the active or first electrode 20 and the conductive membrane 18 in contact therewith, the medicament or carrier therefor in the matrix 15, the individual's body and the ground electrode 24. Thus, an electrical current is caused to flow thereby electrokinetically driving the medicament into the targeted treatment site.

To provide broader area coverage for the medicament, and simultaneously to avoid the problems of short-circuiting the electrical current through current pathways of least resistance, a plurality of applicators 11 may be provided, e.g., in sheet form. The applicators are separable to provide groups of applicators for selected area coverage. The area coverage of the applicators 11 is aggregated as dictated by the area of the treatment site and the areas of the individual applicators 11 themselves. Referring to FIG. 2, for example, each applicator may be in the form of a hexagon and a plurality of hexagon-shaped applicators may be provided in sheet form with each applicator being separable by perforations 30. A multi-channel electrode array, e.g., electrodes 32, 34, 36, 38 and 40 coupled to a microprocessor 42 supplies electrical current to the applicators. For example, each electrode may be in electrical contact with one applicator or aligned in rows of applicators 11 as illustrated in FIG. 2. Thus, one electrode may control one applicator or a multiplicity of applicators. Under the control of the microprocessor, individual applicators or lines (electrodes?) of applicators may be powered all at the same time, in a sequence or randomly. In the latter cases, such that not all applicators will receive power at the same time, the total amount of current passing through the administration site is decreased at any one instant of time. This will allow for large surface area multi-channel applications when the electric current is passing across the heart. The microprocessor may also ramp the current supplied to the electrodes up and/or down as a function of time. With the multiplicity of needles in each applicator providing a low resistance channel through the high electrically resistant layer or layers of the skin and essentially bypassing the high resistance layer(s), the medicament is electrokinetically driven into the target site along a multiplicity of low resistance paths thereby precluding shorting of the electrical current among the various paths. Consequently, by using large area pads consisting of a plurality of applicators 11 overlying a treatment site and supplying electrical current via the multi-channel electrode array, medicament is electrokinetically driven into the targeted treatment site bypassing the one or more skin layers of higher electrical resistance.

Although the example embodiment uses a microprocessor to control currents supplied to the electrodes, other types of processing may be used such as application specific integrated circuits, programmable logic arrays, and the like.

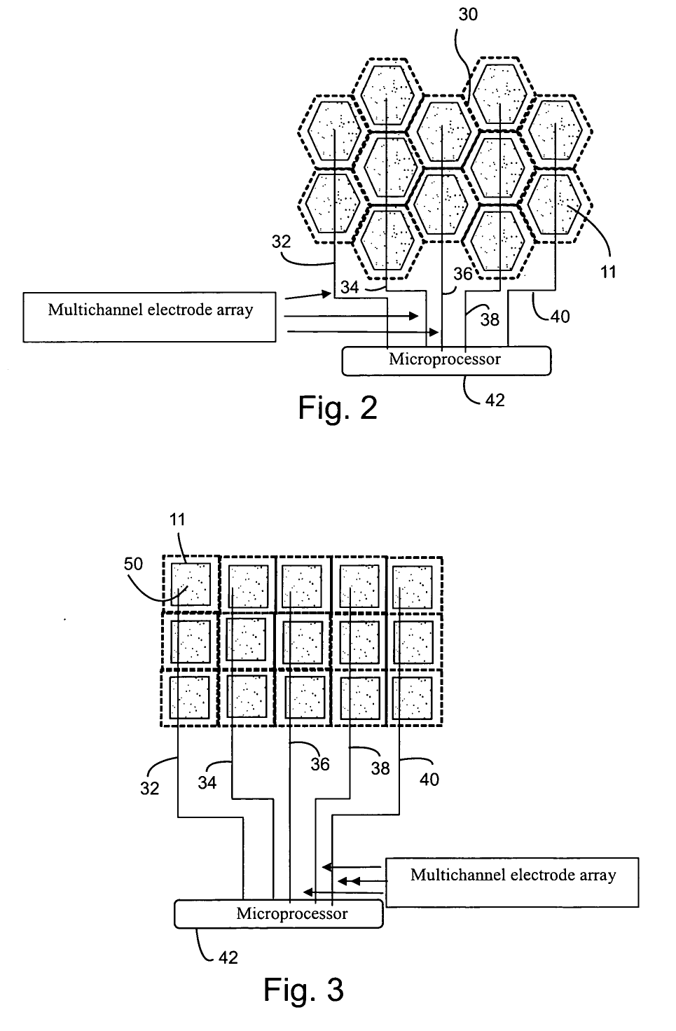

Referring to FIG. 3, there is illustrated a further embodiment of the system wherein the applicators 11 are shaped in rectangles 50, preferably squares, and connected in line by a multi-channel electrode array with the microprocessor. It will be appreciated that shapes of applicators 11 other than hexagonal, rectangular, or square may be provided, e.g., circular. The system of FIG. 3 delivers the medicament to the targeted site similarly as in FIG. 2. It will be appreciated that any number of applicators may be aggregated to form the large area applicator pad and thus may be in any size or configuration conformed to the targeted treatment site.

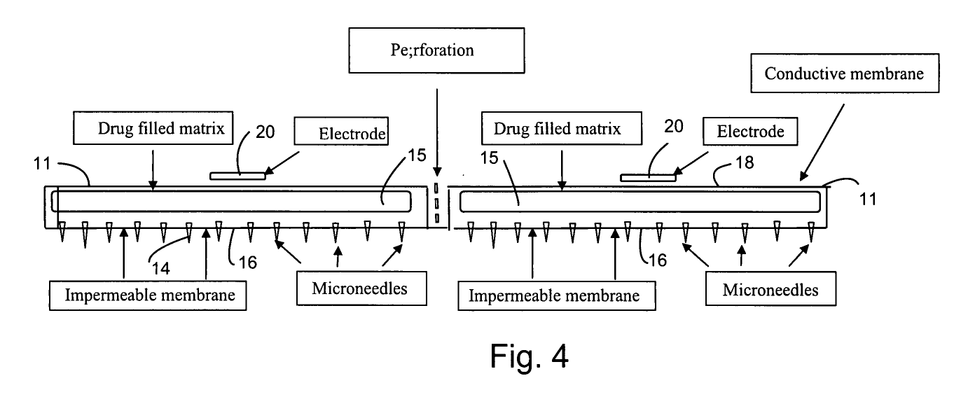

FIG. 4 is a schematic representation of multiple applicators which may form part of the sheet of applicators of FIGS. 2 and/or 3. Two applicators 11 are illustrated in side by side relation and form part of the large area array of the electrokinetic medicament delivery system. Each applicator 11 is illustrated with a separate active electrode 20 which may form part of a reusable device in contrast to the disposable applicator. For example, where multiple active electrodes are provided on the tip of an electrokinetic device, such as the finger mounted device of U.S. Pat. No. 6,792,306, or the hand-held pen-like device of U.S. Reissue Pat. No. RE37796, the applicators are oriented such that when attached to those devices the active electrodes electrically connect with the individual electrodes of the multi-channel electrode array. Thus, the applicator may be attached to the device only in one orientation where this electrical connection can be accomplished. For example, by sizing or configuring the perimeter of the applicators to the same configuration of the perimeter of the device, the active electrodes, i.e., the multi-channel electrodes are automatically aligned with the conductive membrane of the applicators, respectively. Further, disposable applicators may have integral etched electrodes leading to a connector which plugs in or receives a plug from a control unit housing the microprocessor that controls the electrical current flowing through each electrode and applicator.

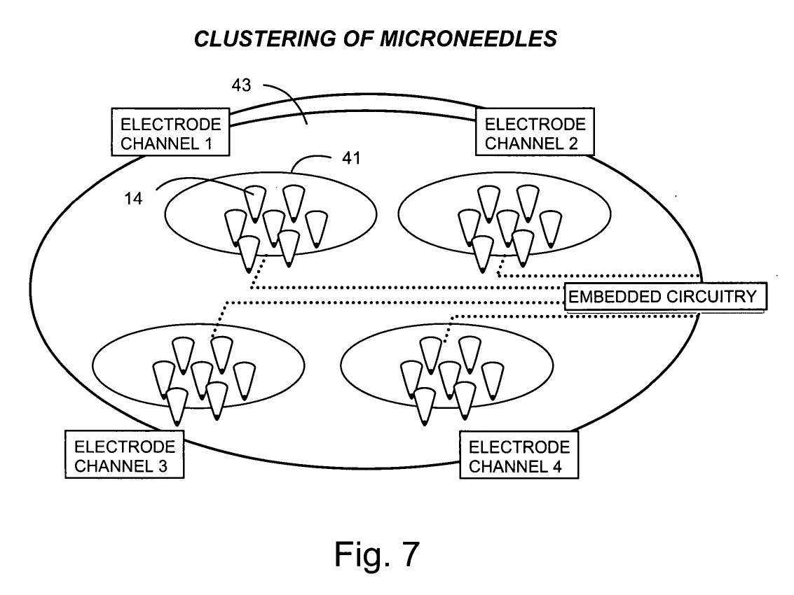

Referring to FIG. 7, and as evident from the foregoing, the micro-needles 14 may be provided in clusters 41 carried by a substrate 43. The micro-needles 14 of each cluster are provided with an individual electrode channel by way of electrodes imbedded within the substrate 43 supplying current to each of the needles of the cluster.

Referring to FIG. 8, the applicator 11 may be flexible for conformance with the contours of the individual's skin at the treatment site. The applicator 60 may include a flexible electrode 62 overlying a non-woven or woven fabric 64 containing, e.g., saturated with the medicament. Underlying the woven or non-woven material is a substrate, for example formed of silica. Micro-needles 68 are carried by the substrate with orifices of the micro-needles in communication with the medicament or conductive carrier therefor in the woven or non-woven material. As illustrated, the micro-needles 68 may have offset orifices 70 opening through the sides of the micro-needles or the orifices may take any one of the sizes and/or configurations of micro-needles described and illustrated with respect to FIG. 5. The flexible nature of the applicator of FIG. 8 enables it to be applied more readily to contoured surfaces along the individual's skin and may be supplied as a single applicator or as a multiplicity of applicators in sheet form, for example, as previously described. The applicator of FIG. 8 operates to electrokinetically deliver the medicament to the treatment site similarly as described in the previous embodiments.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A device for delivering a medicament to a treatment site underlying a layer of an individual's skin having a higher electrically resistant layer than a skin layer at the treatment site, comprising:
an applicator for overlying the treatment site and the electrically resistant skin layer, said applicator having a plurality of needles projecting from a first surface thereof for penetrating the electrically resistant layer of the individual's skin, said needles and said surface being formed of a non-electrically conductive material;
a matrix carried by said applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site;
said applicator having a second surface formed of electrically conductive material.

2. A device according to claim 1, wherein said surfaces lie on respective opposite sides of the applicator and encapsulate the medicament or the medicament and carrier therefor.

3. A device according to claim 1, wherein the needles comprise non-electrically-conductive micro-needles.

4. A device according to claim 3, wherein the needles are formed of a thermoplastic material.

5. A device according to claim 1, wherein the applicator is formed of a flexible material for conformance to the contour of the individual's skin.

6. A device according to claim 1, wherein the needles comprise micro-needles, said micro-needles being formed of electrically conductive material having non-electrically-conductive coatings applied thereto.

7. A device according to claim 1, wherein the needles comprise micro-needles formed of a sintered material.

8. A system according to claim 1, wherein said needles comprise non-electrically conductive micro-needles, said first surface including an impermeable, non-electrically-conductive membrane carrying said micro-needles, said second surface comprising an electrically conductive impermeable membrane on an opposite side of said application from said first surface, margins of said applicator being at least in part formed of a non-electrically conductive material.

9. A system for delivering a medicament to a treatment site underlying an electrically resistant layer of an individual's skin, comprising:
an applicator for overlying the treatment site and the electrically resistant skin layer, said applicator having a plurality of needles projecting from one side thereof for penetrating the electrically resistant layer of the individual's skin;
a matrix carried by said applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site;
a first electrode for electrical connection with a power source;
whereby, upon application of the applicator to the individual's skin overlying the treatment site and connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow for electrokinetically driving the medicament or the medicament and the electrical carrier therefor through the needle orifices into the treatment site bypassing the electrically resistant layer of the individual's skin.

10. A system according to claim 9, wherein the needles comprise non-electrically-conductive micro-needles.

11. A system according to claim 9, wherein the needles are formed of a thermoplastic material.

12. A system according to claim 9, wherein the density of the needles carried by the applicator lies in a range of 1 to 1,000 per sq. cm.

13. A system according to claim 9, wherein the needles comprise micro-needles and each needle has a length to width ratio at a base of the needle in a range of about 0.5 to 2.0.

14. A system according to claim 9, wherein the needles comprise micro-needles, each orifice through the needle having a diameter in a range of 25 to 200 microns.

15. A system according to claim 9, wherein the applicator and the first electrode are separable from one another.

16. A system according to claim 9, wherein the applicator is formed of a flexible material for conformance to variations in contour of the individual's skin.

17. A system according to claim 9, wherein the needles comprise micro-needles, said micro-needles being formed of metal and having non-electrically-conductive coatings.

18. A system according to claim 9, wherein the needles comprise micro-needles formed of a sintered material.

19. A system according to claim 9, wherein said applicator includes an impermeable, non-electrically-conductive membrane carrying said needles.

20. A system according to claim 19, wherein said needles are formed of a non-electrically-conductive material.

21. A system according to claim 20, wherein said applicator includes an electrically conductive membrane on a side of the applicator remote from the impermeable membrane.

22. A system for delivering a medicament to a treatment site underlying an electrically resistant layer of an individual's skin, comprising:
a power source;
an applicator for overlying the treatment site and the electrically resistant skin layer, said applicator having a plurality of needles projecting from one side thereof for penetrating the electrically resistant layer of the individual's skin;
a matrix carried by said applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site;
a first electrode carried by said applicator in electrical connection with said power source;
a second electrode in electrical connection with the power source;
whereby, upon application of the applicator to the individual's skin overlying the treatment site and electrical connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow to electrokinetically drive the medicament or the medicament and the electrical carrier therefor through the needle orifices into the treatment site bypassing the electrically resistant layer of the individual's skin.

23. A system according to claim 22, wherein the needles comprise non-electrically-conductive micro-needles.

24. A system according to claim 22, wherein the needles are formed of a thermoplastic material.

25. A system according to claim 22, wherein the density of the needles carried by the applicator lies in a range of 1 to 1,000 per sq. cm.

26. A system according to claim 22, wherein the needles comprise micro-needles and each needle has a length to width ratio at a base of the needle in a range of about 0.5 to 2.0.

27. A system according to claim 22, wherein the needles comprise micro-needles, each orifice through the needle having a diameter in a range of 25 to 200 microns.

28. A system according to claim 22, wherein the applicator and the first electrode are separable from one another.

29. A system according to claim 22, wherein the applicator and first electrode are formed of a flexible material for conformance to the contours of the individual's skin.

30. A system according to claim 22, wherein the needles comprise micro-needles, said micro-needles being formed of metal and having non-electrically-conductive coatings.

31. A system according to claim 30, wherein the needles comprise micro-needles formed of a sintered material.

32. A system according to claim 30, wherein said applicator includes an impermeable, non-electrically-conductive membrane carrying said needles.

33. A system according to claim 32, wherein said needles are formed of a non-electrically-conductive material.

34. A system according to claim 33, wherein said applicator includes an electrically conductive membrane on a side of the applicator remote from the impermeable membrane.

35. A system for delivering a medicament to a treatment site underlying an electrically resistant layer of an individual's skin, comprising:
a sheet of discrete applicators selectively separable from one another enabling one or more of the applicators to overlie the treatment site and the electrically resistant skin layer, each said applicator having a plurality of needles projecting from one side thereof for penetrating the electrically resistant layer of the individual's skin;
a matrix carried by each said applicator for containing the medicament or the medicament and an electrical carrier therefor, the needles of each applicator having one or more orifices in communication with the medicament or the medicament and the electrical carrier therefor contained in the matrix and opening at locations spaced from the matrix for delivering the medicament to the treatment site;
a first electrode carried by each applicator for electrical connection with a power source;
whereby, upon application of one or more of the applicators to the individual's skin overlying the treatment site and connection to the power source and a second electrode in electrical connection with the power source enabling completion of an electrical circuit through the first one or more electrodes, the medicament or the electrical carrier therefor of the one or more applicators, a portion of the individual's body, the second electrode and the power source, the system enables an electrical current to flow for electrokinetically driving the medicament or the medicament and the electrical carrier therefor through the needle orifices of the one or more applicators into the treatment site bypassing the electrically resistant layer of the individual's skin.

36. A system according to claim 35, wherein the needles comprise non-electrically-conductive micro-needles.

37. A system according to claim 35, wherein the needles are formed of a thermoplastic material.

38. A system according to claim 35, wherein each applicator and the first electrode carried thereby are separable from one another.

39. A system according to claim 35, wherein the one or more applicators are formed of a flexible material for conformance to the contours of the individual's skin.

40. A system according to claim 35, wherein the needles comprise micro-needles, said micro-needles being formed of metal and having non-electrically-conductive coatings.

41. A system according to claim 35, wherein the needles comprise micro-needles formed of a sintered material.

42. A system according to claim 35, wherein said applicator includes an impermeable, non-electrically-conductive membrane carrying said needles.

43. A system according to claim 42, wherein said needles are formed of a non-electrically-conductive material.

44. A system according to claim 43, wherein said applicator includes an electrically conductive membrane on a side of the applicator remote from the impermeable membrane.

45. A method for delivering medicament to a treatment site underlying an electrically resistant layer of an individual's skin, comprising the steps of:
applying a plurality of micro-needles to the individual's skin to penetrate the electrically resistant layer of the individual's skin; and
electrokinetically driving the medicament or the medicament and an electrical carrier therefor through the micro-needles into the treatment site bypassing the electrically resistant layer of the individual's skin.

46. A method according to claim 45, including providing the micro-needles in discrete applicators, providing one or more electrodes for the respective applicators and one or more channels connected to a power source and to one or more of said electrodes to electrokinetically drive the medicament or carrier therefor in said applicators in a large distribution area substantially corresponding to the area of the individual's skin overlaid by the applicators.

47. A method according to claim 45, including providing the micro-needle carrying applicators in a sheet of discrete applicators each having at least one electrode, separating at least one applicator from the sheet of applicators to overlie the treatment site.

48. A method according to claim 45, including providing the plurality of micro-needles in discrete applicators, providing at least one electrode for each applicator and electrically connecting the electrodes and a power source.
